(19) United States
(12) Patent Application Publication
(10) Pub. No.: US 2002/0128226 A1
(43) Pub. Date: Sep. 12, 2002

(54) Enoxaparin and methods of its use

(76) Inventors: Christopher Kern, Edenkoben (DE); Christine Hoerber, Mainz (DE); Eckart Bartnik, Wiesbaden-Delkenheim (DE); Philipp Haus-Seuffert, Frankfurt (DE)

Correspondence Address: Finnegan, Henderson, Farabow,, Garrett & Dunner, L.L.P., 1300 I Street, N.W., Washington, DC, 20005-3315, US

(21) Appl. No.: 10/014,472

(22) Filed: Dec. 14, 2001

(30) Foreign Application Priority Data
Dec. 16, 2000 (DE) 10063006.5-41

(51) Int. Cl. A61K 31/727

(52) U.S. Cl. 514/56

(57) ABSTRACT

This present invention discloses inhibitors of matrix metalloproteinases, and novel methods of their use. The inhibitors may be useful for treating conditions that involve enhanced activity of at least one of the matrix metalloproteinases neutrophil collagenase (MMP-8), aggrecanase, hADAMTS1, and gelatinase A (MMP-2). Such disorders may include, but are not limited to, degenerative joint disorders (e.g., osteoarthroses), spondyloses, chondrolysis associated with joint trauma or prolonged joint immobilization (often occurring after meniscus or patellar injuries or ligament tears), connective tissue disorders (e.g., collagenoses), wound healing disturbances, periodontal disorders, chronic disorders of the locomotor system (e.g., inflammatory, immunologically, or metabolism-related acute and chronic arthritides), arthropathies, myalgias, or disturbances of bone metabolism.

### FIELD OF THE INVENTION

This invention relates to inhibitors of matrix metalloproteinases, and methods of their use. The inhibitors are useful for treating conditions that exhibit enhanced activity of matrix metalloproteinases.

### BACKGROUND

Enoxaparin is a known compound that has reportedly been employed for the treatment of thromboses (U.S. Pat. No. 5,389,618). Enoxaparin-Na is the sodium salt of low molecular weight heparin. It is obtained by alkaline depolymerization of the benzyl ester derivatives of heparin from porcine intestinal mucosa. Most of the polymerized molecules have a 4-enopyranose-uronate structure at the nonreducing end of their chain. The average molecular mass of these depolymerized molecules is about 4,500 daltons. About 12% (w/w) to 20% (w/w) of these molecules are smaller than 2,000 daltons. About 68% (w/w) to 88% (w/w) of the molecules have a size between 2,000 and 8,000 daltons (as compared with the European Pharmacopoeia calibration reference standard for low molecular weight heparins). The degree of sulfation is 2 per disaccharide unit.

The enoxaparin polysaccharide chain is composed, like heparin, of alternating units of sulfated glucosamines and uronic acids linked by glycosidic bonds. The structure of enoxaparin differs from heparin, however, in that the depolymerization process results in a double bond at the nonreducing end of the chain. Enoxaparin can be distinguished from heparin by UV spectroscopy and ¹³C nuclear magnetic resonance spectrum analysis, which show the double bond in the terminal ring; the compounds are also distinguishable by high performance size exclusion chromatography.

In the pathological state of osteoarthrosis, degradation of aggrecan, the major proteoglycan of the articular cartilage, represents a very early and crucial event. The pathological loss of aggrecan is caused by proteolytic cleavages in its interglobular domain. Amino acid sequence analyses of proteoglycan metabolites isolated from the synovial fluid of patients suffering from joint damage, osteoarthrosis, or an inflammatory joint disorder have shown that a proteolytic cleavage between amino acids Glu³⁷³ and Ala³⁷⁴ in the interglobular domain of human aggrecan (Lohmander, et al., Arthritis Rheum., 36: 1214-1222 (1993)). The proteolytic activity responsible for this cleavage is referred to as “aggrecanase,” and may be assigned to the superfamily of metalloproteinases (MP) or matrix metalloproteinases (MMP).

Zinc is essential in the catalytically active site of metalloproteinases. MMPs cleave collagen, laminin, proteoglycans, elastin, or gelatin under physiological conditions. Therefore, they play an important role in bone and connective tissue. A large number of different inhibitors of MMPs are known (see, e.g., EP 0 606 046; WO 94/28889). However, the known inhibitors of MMPs frequently have a significant disadvantage. They lack specificity for any particular class of MMPs. Instead, most MMP inhibitors inhibit a plurality of MMPs simultaneously.

Thus, there exists a need in the art for inhibitors of MMPs that have more narrowly defined specificities to better treat or prevent specific disorders.

### SUMMARY OF THE INVENTION

The present invention is directed to compounds that are useful for inhibiting matrix metalloproteinases, and methods of their use. In one embodiment, the compound is enoxaparin. Enoxaparin is useful for inhibiting mettalloproteinases, such as neutrophil collagenase (MMP-8), aggrecanase, hADAMTS1, and gelatinase A (MMP-2).

In one embodiment, enoxaparin may be provided in a pharmaceutically acceptable composition to treat or prevent disorders that display an enhanced activity of at least one of matrix metalloproteinases, such as neutrophil collagenase (MMP-8), aggrecanase, hADAMTS1 and gelatinase A (MMP-2). Such disorders may include, but are not limited to, degenerative joint disorders (e.g., osteoarthroses), spondyloses, chondrolysis associated with joint trauma or prolonged joint immobilization (often occurring after meniscus or patellar injuries or ligament tears), connective tissue disorders (e.g., collagenoses), wound healing disturbances, periodontal disorders, chronic disorders of the locomotor system (e.g., inflammatory, immunologically, or metabolism-related acute and chronic arthritides), arthropathies, myalgias, or disturbances of bone metabolism.

Enoxaparin may be administered by a number of different routes. Suitable routes of administration include, but are not limited to, subcutaneous, intraarticular, intraperitoneal, or intravenous injection. Administration may also be rectal, oral, inhalational, or transdermal. In one embodiment, a solution of enoxaparin is administered by intraarticular injection. The dosage amount will typically range from about 5 μg to about 200 mg of enoxaparin, in a pharmaceutically acceptable composition. In another embodiment, about 10 μg to about 40 mg of enoxaparin is administered.

The present invention is also directed to a method of identifying inhibitors of aggrecanase activity. The method generally comprises incubating a target substrate molecule and a suspected inhibitor with a metalloproteinase that exhibits aggrecanase activity; and identifying the presence or absence of neoepitopes from the substrate molecule to determine if the aggrecanase activity of the metalloproteinase is active against the target substrate, or if the activity is inhibited by the presence of the suspected inhibitor. In one embodiment, ADAMTS1 is the metalloproteinase used in this method to detect inhibitors of aggrecanase activity. The metalloproteinase (e.g., ADAMTS1) may further be incorporated into a test kit to identify inhibitors of aggrecanase activity.

### DETAILED DESCRIPTION OF THE INVENTION

The present invention relates to inhibitors of specific metalloproteinases, which are useful in treating disorders. It has now been found that enoxaparin, employed according to the invention, is a strong inhibitor of matrix metalloproteinases including neutrophil collagenase (MMP-8), aggrecanase, hADAMTS1, and gelatinase A (MMP-2), but does not effectively inhibit MMP -1, -3, -13 and -14.

The invention therefore relates to the use of enoxaparin to treat or prevent disorders that exhibit an enhanced activity of at least one of the matrix metalloproteinases neutrophil collagenase (MMP-8), aggrecanase, hADAMTS1, and gelatinase A (MMP-2).

Due to its pharmacological properties in inhibiting specific metalloproteinases, enoxaparin may be useful for treating or preventing any disorder displaying an enhanced activity of any of the matrix metalloproteinases MMP 8, aggrecanase, hADAMTS1 and MMP 2. These include degenerative joint disorders (e.g., osteoarthroses), spondyloses, and chondrolysis after joint trauma or prolonged joint immobilization (often occurring after meniscus or patellar injuries or ligament tears). They also include connective tissue disorders (such as collagenoses), periodontal disorders, wound healing disturbances, chronic disorders of the locomotor system (such as inflammatory, immunologically or metabolism-related acute and chronic arthritides), arthropathies, myalgias, and disturbances of bone metabolism.

Enoxaparin may be provided in a pharmaceutical composition. Enoxaparin and physiologically tolerated salts of enoxaparin are known and can be prepared as described, for example, in U.S. Pat. No. 5,389,618. Enoxaparin may be administered subcutaneously, intraarticularly, intraperitoneally, or intravenously (e.g., injection). In one embodiment, enoxaparin is administered intraperitoneally. In other embodiments, intraarticular injection may be used. Rectal, oral, inhalational or transdermal administration is also possible.

The invention also relates to a process for producing a pharmaceutical composition, which comprises converting enoxaparin into a suitable dosage form with a pharmaceutically acceptable and physiologically tolerated carrier. Where appropriate, other pharmaceutically acceptable active ingredients, additives, or excipients may be added to the composition.

The pharmaceutical compositions may be produced and administered in dosage units, each unit containing a particular dose of enoxaparin as an active ingredient. In the case of solutions for injection (e.g., in ampoule form), this dose may be from about 5 μg to about 200 mg, and more usually from about 10 μg to 40 mg.

The daily dosage indicated for the treatment of an adult patient weighing about 70 kg is from about 10 μg to about 500 mg of active ingredient, generally from about 20 mg to about 100 mg. However, in some circumstances, higher or lower daily doses may also be appropriate. The daily dose may be administered in a number of different ways. For example, the dose may be administered once a day in the form of a single dosage unit. Alternatively, the dose may be provided in a plurality of smaller dosage units given repeatedly at defined intervals over a period of time.

The present invention is also directed to a method of identifying inhibitors of aggrecanase activity. The method generally comprises incubating a target substrate molecule and a suspected inhibitor with a metalloproteinase that exhibits aggrecanase activity; and identifying the presence or absence of neoepitopes from the substrate molecule to determine if the aggrecanase activity of the metalloproteinase is active against the target substrate, or if the activity is inhibited by the presence of the suspected inhibitor. In one embodiment, ADAMTS1 is the metalloproteinase used in this method to detect inhibitors of aggrecanase activity. The metalloproteinase (e.g., ADAMTS1) may further be incorporated into a test kit to identify inhibitors of aggrecanase activity.

### EXAMPLES

### Example 1

### Effect of enoxaparin on the aggrecanase of porcine chondrocytes

To generate aggrecanase activity, porcine chondrocytes were stimulated with 10 ng/ml IL-1α for 4 days (Hughes, C. E., Little, C. B., Buettner, F. H., Bartnik, E., Caterson, B., “Differential expression of aggrecanase and matrix metallo-proteinase activity in chondrocytes isolated from bovine and porcine articular cartilage,” J. Biol. Chem., 273: 30576-30582 (1998)). In a 96-well cell culture plate, 200 μl of the chondrocyte supernatant containing aggrecanase activity were mixed with 100 μl of (cell culture medium/buffer) DMEM per well. 5 μl of enoxaparin, dissolved in an appropriate concentration in H₂O, were added as an inhibitor one hour before addition of 5 μg of rAgg1mut substrate (Buttner et al., Biochem. J., 333: 159-165 (1998)). The mixture was incubated at 37° C. for 17 h and then transferred into an ELISA plate in order to detect the neoepitopes generated by the aggrecanase activity (Büttner et al., Trans. Orthop. Res. Soc., 23: 916 (1998)) with the antibody BC-3 (Hughes et al., Biochem. J., 305, 799-804 (1995)). The enzymatic activity of a representative experiment, expressed as the average BC-3 signal (extinction), is provided in Table 1.

TABLE 1
Digestion of rAgg1mut by porcine chondrocyte aggrecanase:
Inhibition by enoxaparin

| Enoxaparin | Average BC-3 | Standard |
|---|---|---|
| (μg/ml) | signal n = 2 | deviation |
| 0 | 1.15 | 0.031 |
| 0.0167 | 1.24 | 0.020 |
| 0.167 | 1.13 | 0.031 |
| 1.67 | 1.05 | 0.027 |
| 16.7 | 0.70 | 0.049 |
| 167 | 0.33 | 0.041 |
| 1670 | 0.44 | 0.027 |
| 16700 | 0.27 | 0.063 |
| no aggrecanase and no enoxaparin | 0.06 | 0.002 |
| no aggrecanase and 16700 μg/ml enoxaparin | 0.07 | 0.019 |

The IC₅₀ was determined to be about 80 μg/ml enoxaparin using this experimental approach. As the results indicate, enoxaparin inhibits the aggrecanase activity of porcine chondrocytes in the digestion of the substrate rAgg1mut.

### Example 2

### Effect of enoxaparin on the aggrecanase of human de-differentiated chondrocytes

To generate aggrecanase activity, 50,000 de-differentiated human chondrocytes were stimulated in a 96-well cell culture plate with 0.01 ng/ml IL-1α and 3 U of TNFα in 200 μl of DMEM/F12 medium (1:1) per well for 47 h. In a 96-well cell culture plate, 200 μl of the chondrocyte supernatant containing aggrecanase activity were mixed with 5 μl of enoxaparin (Clexane), dissolved in an appropriate concentration in H₂O, as an inhibitor an hour before addition of substrate (2.5 μg of rAgg1mut). The mixture was incubated at 37° C. for 4 h and then transferred into an ELISA plate in order to detect the neoepitopes generated by aggrecanase activity with the antibody BC-3. The enzymatic activity from a representative experiment, expressed as the BC-3 signal (extinction), is provided in Table 2.

TABLE 2

| | | | Standard | Standard |
|---|---|---|---|---|
| Enoxaparin | Clexane 40 | Clexane 20 | deviation | deviation |
| μg/ml | n = 2 | n = 2 | (Clex 40) | (Clex 20) |
| 0 | 1.20 | | 0.043 | |
| 0.0167 | 1.13 | 1.05 | 0.091 | 0.017 |
| 0.167 | 1.0 | 0.97 | 0.025 | 0.044 |
| 1.67 | 0.99 | 0.92 | 0.064 | 0.009 |
| 16.7 | 0.95 | 0.87 | 0.037 | 0.048 |
| 167 | 0.72 | 0.72 | 0.043 | 0.002 |
| 1670 | 0.29 | 0.42 | 0.007 | 0.084 |
| no aggrecanase and | 0.41 | | 0.016 | |
| no enoxaparin | | | | |
| no aggrecanase and | 0.19 | 0.22 | | |
| 1670 μg/ml | | | | |
| enoxaparin | | | | |

The IC₅₀ was determined to be about 200 μg/ml enoxaparin in this experiment. As the results demonstrate, enoxaparin inhibits the aggrecanase activity of de-differentiated human chondrocytes in the digestion of the substrate rAgg1mut.

### Example 3

### Effect of enoxaparin on the aggrecanase activity of recombinant human ADAMTS1 protein

To generate the aggrecanase activity of human ADAMTS1, 293 cells were transfected with an ADAMTS1 expression plasmid by the calcium phosphate method. An expression plasmid was constructed that harbors the coding sequence of the human ADAMTS1 gene, followed by an inserted C-terminal FLAG tag. The gene was placed under the control of the CMV promoter. The transfection supernatant was passed through an M2 (anti-FLAG) antibody agarose column. The hADAMTS1 was bound to the M2 antibody via its FLAG tag. The recombinant hADAMTS1 was then eluted from the M2 antibody column with free FLAG peptide. The human ADAMTS1 that was partially purified in this manner was employed in the aggrecanase assay described below.

In a 96-well cell culture plate, 10 μl of eluate containing recombinant human ADAMTS1 and 300 μl of DMEM with 5 μl of enoxaparin (Clexane) inhibitor (dissolved in an appropriate concentration in H₂O) were mixed one hour before the addition of substrate (1 μg of rAgg1mut). The mixture was incubated at 37° C. for 4 h and then transferred into an ELISA plate in order to detect the neoepitopes generated by the aggrecanase activity with the antibody BC-3. The enzymatic activity of the representative experiment, expressed as the average BC-3 signal (extinction), is shown in Table 3.

TABLE 3

| Enoxaparin | Average BC-3 |
|---|---|
| μg/ml | signal n = 2 |
| 0 | 1.36 |
| 0.0167 | 1.35 |
| 0.167 | 1.34 |
| 1.67 | 1.32 |
| 16.7 | 0.87 |
| 167 | 0.27 |
| 1670 | 0.23 |
| no hADAMTS1/no enoxaparin | 0.11 |

The IC₅₀ was determined to be about 25 μg/ml enoxaparin in this assay. As the results indicate, enoxaparin inhibits the aggrecanase activity of recombinant human ADAMTS1 in the digestion of the substrate rAgg1 mut.

### Example 4

### Enoxaparin does not inhibit the catalytic domain of MMP-3 in digesting recombinant substrate rAgg1mut

In a 96-well cell culture plate, 31.3 μl of recombinant human MMP-3 (catalytic domain, G98-P273, prepared by the method of Ye et al., Biochemistry, 31:11231-11235 (1992)) in MMP-3 digestion buffer (0.1 M MES; 0.1 M NaCl; 0.01 M CaCl₂; 0.5% Brij; pH 6.0) was mixed with 5 μl of enoxaparin (Clexane) inhibitor, dissolved in an appropriate concentration in H₂O, were mixed one hour before the addition of substrate (5 μg of rAgg1mut). The mixture was incubated at 37° C. for 8 h and then transferred to an ELISA plate in order to detect the neoepitopes generated by MMP-3 activity (cleavage at amino acids N341-F342) with the antibody BC-14 (Hughes et al., Biochem. J., 305:799-804 (1995)). Table 4 shows the results of a representative experiment.

TABLE 4

| Enoxaparin | Average BG-14 | Standard |
|---|---|---|
| μg/ml | signal n = 2 | deviation |
| 0 | 0.97 | 0.044 |
| 0.0167 | 1.02 | 0.004 |
| 1.67 | 1.03 | 0.075 |
| 167 | 0.96 | 0.019 |
| 16700 | 0.94 | 0.042 |
| no MMP-3 and no enoxaparin | 0.14 | 0.057 |
| no MMP-3 and 16700 μg/ml enoxaparin | 0.12 | 0.050 |

As the results indicate, enoxaparin showed no effect on the catalytic domain of MMP-3 on digestion of the recombinant substrate rAgg1 mut.

### Example 5

### Preparation and determination of the enzymatic activity of gelatinase A (MMP-2) and neutrophil collagenase (MMP-8)

Gelatinase A (MMP-2) and neutrophil collagenase (MMP-8) were purchased from Roche and Biocon, respectively. To measure the enzymatic activity or the enzyme-inhibitory effect, 10 μl of enzyme-containing buffer solution were incubated with 10 μl of H₂O which contains the enzyme inhibitor for 15 minutes. MMP-2 (20 mUnit) or MMP-8 (20 ng) were incubated by the method described by Knight (Knight et al., FEBS Letters, 296:263 (1992)) with 10 μl of a 10% strength (v/v) aqueous dimethyl sulfoxide solution containing 0.1 mmol/l of a fluorogenic substrate (7-methoxycoumarin-4-yl)acetyl-Pro-Leu-Gly-Leu-3-(2′,4′-dinitrophenyl)-L-2,3-diaminopropionyl-Ala-Arg-NH₂ (Bachem, Heidelberg, Germany). The progress of the enzymatic reaction was followed by fluorescence spectroscopy (328 nm (ex)/393 nm (em)). The fluorescence was measured for 15 minutes. The initial rate of the enzymatic reaction was measured without addition of inhibitor, and the resulting values were defined as 100% activity.

The compounds tested included unfractionated heparin (UF) with a molecular weight of about 15,000 daltons, a 3,000 dalton (LMW) heparin fraction (both obtainable from Sigma), and enoxaparin. The MMP activities of MMP-2 and MMP-8 were assayed with each test compound.

The inhibitiors listed in Table 5 were each measured at a concentration of 1 μg/ml and were compared with a control without inhibitor (100%).

TABLE 5

| | MMP-2 | MMP-8 |
|---|---|---|
| Compound | inhibition (%) | inhibition (%) |
| UF heparin | 41 | 1 |
| LMW heparin | 27 | 8 |
| Enoxaparin | 70 | 28 |

We claim:

1. A method of treating or preventing a disorder comprising administering to a subject a therapeutically effective amount of enoxaparin, wherein the disorder displays enhanced activity of at least one of the matrix metalloproteinases neutrophil collagenase (MMP-8), aggrecanase, hADAMTS1, and gelatinase A (MMP-2).

2. The method of claim 1, wherein the disorder is a degenerative joint disorder, connective tissue disorder, wound healing disturbance, periodontal disorder, disorder of the locomotor system, or disturbance of bone metabolism.

3. The method of claim 2, wherein the disorder is osteoarthroses, spondyloses, chondrolysis, collagenoses, inflammatory, immunological or metabolism-related acute and chronic arthritides, arthropathies, or myalgias.

4. The method of claim 1, wherein the enoxaparin is administered by subcutaneous, intraarticular, intraperitoneal or intravenous injection.

5. The method of claim 4, wherein the enoxaparin is administered by intraarticular injection.

6. The method of claim 1, wherein the dose of enoxaparin is from about 5 μg to about 200 mg.

7. The method of claim 1, wherein the dose of enoxaparin is from about 10 μg to about 40 mg.

8. The method of claim 1, wherein the enoxaparin is administered rectally, orally, inhalationally, or transdermally.

9. A method of identifying inhibitors of aggrecanase activity comprising mixing ADAMTS1 with a test inhibitor molecule and a substrate, and identifying the presence or absence of neoepitopes generated by the aggrecanase activity of ADAMTS1 after a period of incubation.

10. The method of claim 9, wherein the ADAMTS1 is provided in a test kit.
